(19) United States
(12) Patent Application Publication
(10) Pub. No.: US 2024/0082248 A1
(43) Pub. Date: Mar. 14, 2024

(54) PROCESS FOR PREPARATION OF MAVACAMTEN AND SOLID STATE FORMS THEREOF

(71) Applicant: DR. REDDY'S LABORATORIES LIMITED, Hyderabad (IN)

(72) Inventors: Divya Jyothi KALLEM, Hyderabad (IN); Sharmistha PAL, Bangalore (IN); Srinivas ORUGANTI, Hyderabad (IN); Magesh SAMPATH, Kancheepuram (IN); Kottur Mohan KUMAR, Hyderabad (IN); Saikat SEN, Sonarpur (IN); Arijit MUKHERJEE, 24-Paraganas (IN)

(21) Appl. No.: 18/275,284

(22) PCT Filed: Jan. 31, 2022
(86) PCT No.: PCT/IN2022/050080

§ 371 Date: Aug. 1, 2023

(30) Foreign Application Priority Data
Feb. 1, 2021 (IN) 202141004381
Feb. 16, 2021 (IN) 202141006543
May 13, 2021 (IN) 202141021624
Oct. 5, 2021 (IN) 202141045274

(51) Int. Cl. A61K 31/513; A61K 47/32; A61K 47/38; C07D 239/10

(52) U.S. Cl. CPC A61K 31/513; A61K 47/32; A61K 47/38; C07D 239/10; C07B 2200/13

(57) ABSTRACT

The present application relates to process for preparation of Mavacamten, preparative methods of various crystalline forms of Mavacamten and amorphous form of Mavacamten, its preparative method, and pharmaceutical compositions thereof. The present application also relates to solid dispersions of Mavacamten, their preparative methods and pharmaceutical compositions containing solid dispersions of Mavacamten.

### CROSS-REFERENCE TO RELATED APPLICATIONS

This application claims the benefit of Indian provisional patent application No. 202141004381 filed on 1 Feb. 2021; Indian provisional patent application No. 202141006543 filed on 16 Feb. 2021; Indian provisional patent application No. 202141021624 filed on 13 May 2021; and Indian provisional patent application No. 202141045274 filed on 5 Oct. 2021.

### FIELD OF THE INVENTION

The present application relates to processes for preparation of Mavacamten, solid state forms of Mavacamten, and pharmaceutical compositions thereof.

### BACKGROUND OF THE INVENTION

The drug compound having the adopted name Mavacamten, has a chemical name 6-(((1S)-1-Phenylethyl)amino)-3-propan-2-yl)-1,2,3,4-tetrahydropyrimidine-2,4-dione, and is represented by the structure of formula I.

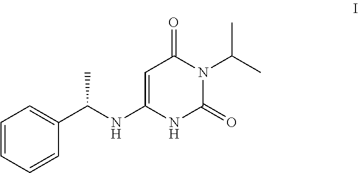 [SMILES: CC(C)n1c(=O)cc(N[C@@H](C)c2ccccc2)[nH]c1=O]

MyoKardia is developing Mavacamten, an allosteric modulator of cardiac myosin that targets aberrant sarcomeres, for the potential oral treatment of genetic cardiomyopathies including obstructive hypertrophic cardiomyopathy (HCM) and-obstructive HCM.

Mavacamten, its synthetic process and its pharmaceutical compositions are described in U.S. Pat. No. 9,181,200 B2 (US '200) and U.S. Pat. No. 9,585,883 B2 (US '883). The process described in US '519 is schematically represented below:

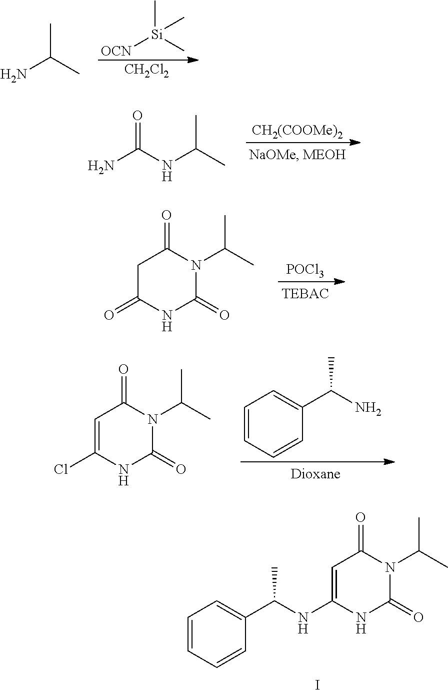

Mavacamten prepared by the synthetic process described in US '200 and US '883 contains about 20% of unreacted starting material, 6-chloro-3-isopropyl pyrimidine-2,4-dione, as impurity. Hence, there remains a need to provide commercially viable and advantageous processes for preparation of pure Mavacamten, and pharmaceutical compositions thereof.

Polymorphism, the occurrence of different crystal forms, is a phenomenon of some molecules and molecular complexes. A single molecule may give rise to a variety of polymorphs having distinct crystal structures and physical properties. Polymorphs in general will have different melting points, thermal behaviors (e.g. measured by thermogravimetric analysis—“TGA”, or differential scanning calorimetry—“DSC”), X-ray powder diffraction (XRPD or powder XRD) pattern, infrared absorption fingerprint, and solid state nuclear magnetic resonance (NMR) spectrum. One or more of these techniques may be used to distinguish different polymorphic forms of a compound.

Discovering new polymorphic forms, hydrates and solvates of a pharmaceutical product can provide materials having desirable processing properties, such as ease of handling, ease of processing, storage stability, and ease of purification or as desirable intermediate crystal forms that facilitate conversion to other polymorphic forms. New polymorphic forms and solvates of a pharmaceutically useful compound or salts thereof can also provide an opportunity to improve the performance characteristics of a pharmaceutical product. It enlarges the repertoire of materials that a formulation scientist has available for formulation optimization, for example by providing a product with different properties, e.g., better processing or handling characteristics, improved dissolution profile, or improved shelf-life. For at least these reasons, there is a need for additional solid forms of Mavacamten.

### SUMMARY OF THE INVENTION

The present application generally relates to process for preparation of Mavacamten, its crystalline forms and solid dispersions, and pharmaceutical compositions thereof.

In one aspect the present application provides a solid dispersion comprising Mavacamten and one or more pharmaceutically acceptable excipient, and process thereof.

In another aspect the present application provides amorphous form of Mavacamten, and process thereof.

In another aspect the present application provides a process for preparation of crystalline Form A of Mavacamten, characterized by a PXRD pattern comprising peaks at about 11.5, 15.6, 17.2, 18.6, 19.9, 22.2, 23.3, 25.5, 29.0 and 31.5±0.2° 2θ, comprising:
a) providing a solution of Mavacamten,
b) adding an anti-solvent to the solution obtained in step (a),
c) optionally, heating the mixture of step (c), and
d) isolating crystalline Form A of Mavacamten.

In another aspect the present application provides a process for preparation of crystalline Form B of Mavacamten characterized by a PXRD pattern comprising peaks at about 8.3, 11.7, 13.2, 15.6, 18.5, 18.7, 19.9, 22.1, 24.4 and 26.8±0.2° 2θ, comprising:
a) providing a solution of Mavacamten,
b) adding the solution obtained in step (a) into water at 0° C., and
c) isolating crystalline Form B of Mavacamten.

In another aspect the present application provides a process for preparation of crystalline Form B of Mavacamten characterized by a PXRD pattern comprising peaks at about 8.3, 11.7, 13.2, 15.6, 18.5, 18.7, 19.9, 22.1, 24.4 and 26.8±0.2° 2θ, comprising slurrying amorphous Mavacamten in water and isolating the crystalline Form B of Mavacamten.

In another aspect the present application provides a process for preparation of crystalline Form C of Mavacamten characterized by a PXRD pattern comprising peaks at about 7.8 and 18.1±0.2° 2θ, comprising:
a) providing a solution of Mavacamten in methanol,
b) optionally, heating the solution obtained in step (a) to 50° C., and
c) isolating crystalline Form C of Mavacamten.

In another aspect the present application provides a process for preparation of crystalline Form D of Mavacamten, characterized by a PXRD pattern comprising peaks at about 11.06, 14.4, 15.5, 16.9 and 19.1±0.2° 2θ, comprising:
a) heating Mavacamten to temperature up to 230° C.;
b) isolating crystalline Form D of Mavacamten.

In another aspect the present application provides a process for preparation of crystalline Form E of Mavacamten, characterized by a PXRD pattern comprising peaks at about 6.39, 9.31, 13.87, 20.08 and 24.81±0.2° 2θ, comprising:
a) providing Mavacamten in one or more of suitable solvents;
b) isolating crystalline Form E of Mavacamten.

In another aspect the present application provides pharmaceutical composition comprising amorphous solid dispersion of Mavacamten and one or more pharmaceutically acceptable carrier.

In another aspect the present application provides pharmaceutical composition comprising amorphous form of mavacamten or any of crystalline forms of Mavacamten prepared by the process described in this application and one or more pharmaceutically acceptable carrier.

### BRIEF DESCRIPTION OF DRAWINGS

FIG. 1 is powder X-ray diffraction (PXRD) pattern of crystalline Form A prepared according to example 3.

FIG. 2 is powder X-ray diffraction (PXRD) pattern of crystalline Form B prepared according to example 4.

FIG. 3 is powder X-ray diffraction (PXRD) pattern of solid dispersion of Mavacamten prepared according to example 5.

FIG. 4 is powder X-ray diffraction (PXRD) pattern of solid dispersion of Mavacamten prepared according to example 6.

FIG. 5 is powder X-ray diffraction (PXRD) pattern of crystalline Form C prepared according to example 7.

FIG. 6 is powder X-ray diffraction (PXRD) pattern of crystalline Form D prepared according to example 8.

FIG. 7 is powder Differential scanning calorimetry (DSC) graph of crystalline Form D prepared according to example 8.

FIG. 8 is powder X-ray diffraction (PXRD) pattern of amorphous form of Mavacamten prepared according to example 9.

FIG. 9 is powder X-ray diffraction (PXRD) pattern of crystalline Form E prepared according to example 11.

FIG. 10 is powder X-ray diffraction (PXRD) pattern of amorphous solid dispersion of Mavacamten with HPMC phthalate prepared according to example 12.

FIG. 11 is powder X-ray diffraction (PXRD) pattern of amorphous solid dispersion of Mavacamten with PVP K-90 prepared according to example 13.

FIG. 12 is powder X-ray diffraction (PXRD) pattern of amorphous solid dispersion of Mavacamten with Eudragit L100-55 according to example 14.

### DETAILED DESCRITPION

The present application relates to process for preparation of pure Mavacamten, solid state forms of Mavacamten, solid dispersions of Mavacamten and their pharmaceutical compositions.

In one aspect the present application provides amorphous solid dispersion comprising Mavacamten and one or more pharmaceutically acceptable excipient.

In another aspect the present application provides a process for preparation of amorphous solid dispersion comprising Mavacamten and one or more pharmaceutically acceptable excipient, the process comprising:
a) providing a solution comprising Mavacamten and one or more pharmaceutically acceptable excipients,
b) removing solvent from the solution obtained in step (a), and
c) recovering amorphous solid dispersion comprising Mavacamten and one or more pharmaceutically acceptable excipients.

Providing a solution in step (a) includes direct use of a reaction mixture containing Mavacamten that is obtained in the course of its synthesis or dissolving Mavacamten and a pharmaceutically acceptable excipient in a solvent or a mixture of solvents. Any physical form of Mavacamten may be used to provide the solution of step (a).

Suitable pharmaceutically acceptable excipients which may be used in step (a) include, but are not limited to: diluents such as starches, pregelatinized starches, lactose, powdered celluloses, microcrystalline celluloses, dicalcium phosphate, tricalcium phosphate, Polyethylene glycol, Copovidone, Soluplus, Silicified microcrystalline cellulose mannitol, sorbitol, sugar and the like; binders such as acacia, guar gum, tragacanth, gelatin, polyvinylpyrrolidones, methacrylic acid copolymer (Eudragit or Eudragit-RLPO), hydroxypropyl celluloses, hydroxypropyl methylcelluloses such as HPMC-Phthalate, HPMC-AS, HPMC-15 CPS; pregelatinized starches and the like; disintegrants such as starches, sodium starch glycolate, pregelatinized starches, crospovidones, croscarmellose sodium, colloidal silicon dioxide and the like; lubricants such as stearic acid, magnesium stearate, zinc stearate and the like; glidants such as colloidal silicon dioxide and the like; solubility or wetting enhancers such as anionic or cationic or neutral surfactants; complex forming agents such as various grades of cyclodextrins and resins; release rate controlling agents such as hydroxypropyl celluloses, hydroxymethyl celluloses, hydroxypropyl methylcelluloses, ethylcelluloses, methylcelluloses, various grades of methyl methacrylates, waxes and the like. Other pharmaceutically acceptable excipients that are of use include but are not limited to film formers, plasticizers, colorants, flavoring agents, sweeteners, viscosity enhancers, preservatives, antioxidants, and the like. A thorough discussion of pharmaceutically acceptable excipients is presented in Remington's Pharmaceutical Sciences (17th ed., Mack Publishing Company) and Remington: The Science and Practice of Pharmacy (21st ed., Lippincott Williams & Wilkins), which are hereby incorporated by reference.

In a preferred embodiment, the pharmaceutically acceptable excipients are PVP-K30, PVP-K90, Copovidone, HPMC phthalate and Eudragit.

Suitable solvent that can be used for dissolving the Mavacamten is dichloromethane, THF, methanol, ethanol, isopropyl alcohol or a mixture thereof. In a specific aspect the solvent used in step (a) is a mixture of methanol and dichloromethane.

After dissolution in step (a), optionally undissolved particles, if any, may be removed suitably by filtration, centrifugation, decantation, and any other known techniques. The solution can be filtered by passing through paper, glass fiber, or other membrane material, or a clarifying agent such as celite. Depending upon the equipment used and the concentration and temperature of the solution, the filtration apparatus may need to be preheated to avoid premature crystallization.

Step (b) involves removing solvent from the solution obtained in step (a). Suitable techniques which can be used for the removal of solvent include but not limited to evaporation, flash evaporation, simple evaporation, rotational drying such as drying using a rotavapor, spray drying, agitated thin-film drying, agitated nutsche filter drying, pressure nutsche filter drying, freeze-drying, filtration or any other technique known in the art.

Step (c) involves recovering amorphous solid dispersion comprising Mavacamten and one or more pharmaceutically acceptable excipient. The said recovery can be achieved by using the processes known in the art.

The solid obtained in step (c) may optionally be further dried. Drying can be carried out in a tray dryer, vacuum oven, air oven, cone vacuum dryer, rotary vacuum dryer, fluidized bed dryer, spin flash dryer, flash dryer, or the like. The drying can be carried out at temperatures of less than about 75° C., less than about 50° C., or any other suitable temperatures; at atmospheric pressure or under a reduced pressure; as long as the Mavacamten is not degraded in its quality. The drying can be carried out for any desired time until the required product quality is achieved. Suitable time for drying can vary from few minutes to several hours for example from about 30 minutes to about 24 or more hours.

When the active ingredient is hygroscopic or the formulation contains a hygroscopic ingredient, and to increase the stability of the amorphous form or a solid dispersion comprising Mavacamten, addition of other carriers such as syloid, methyl cellulose, colloidal silicon dioxide, Eudragit, amorphous silica, micro crystalline cellulose, and the like, in the formulation has been found to be of particular value. Therefore these ingredients may be combined during the preparation of solid dispersion or after the preparation of solid dispersion to control hygroscopicity and to improve stability.

In another aspect, the present application provides a pharmaceutical composition comprising Mavacamten solid dispersion of the present invention and a pharmaceutically acceptable carrier.

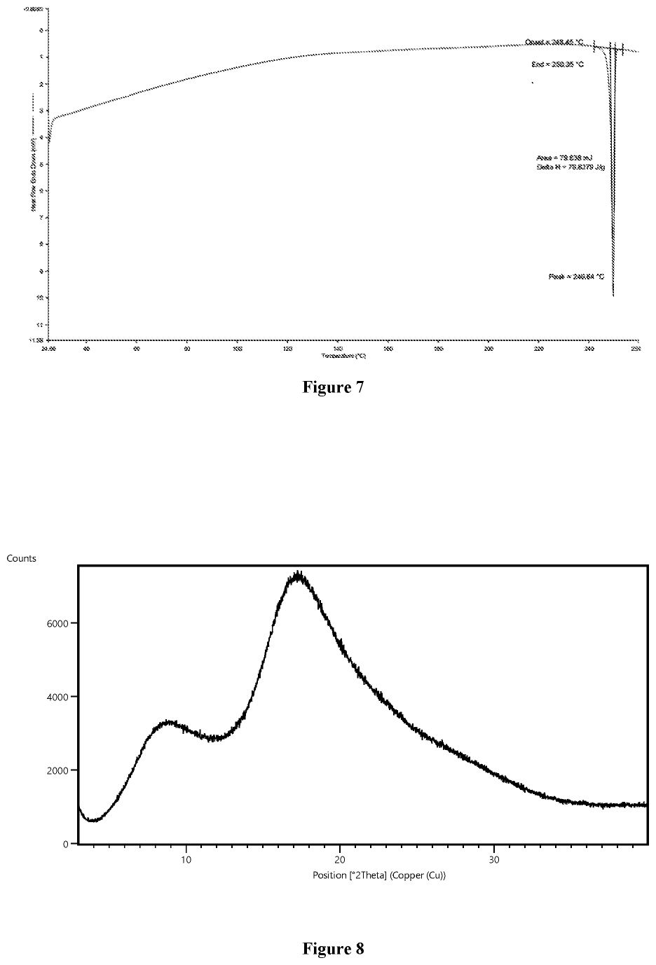

In another aspect the present application provides amorphous form of Mavacamten, characterized by a PXRD pattern as represented by FIG. 8.

In another aspect the present application provides a process for preparation of amorphous form of Mavacamten, comprising:
a) milling Mavacamten,
b) isolating amorphous form of Mavacamten.

In another embodiment, the present application provides a process for preparation of amorphous form of Mavacamten comprising ball milling Form A of Mavacamten.

In another aspect the present application provides a process for preparation of amorphous form of Mavacamten, comprising:
a) providing Mavacamten in one or more of suitable solvents;
b) isolating amorphous form of Mavacamten.

In another aspect the present application provides pharmaceutical compositions comprising amorphous form of Mavacamten described in this application and one or more pharmaceutically acceptable excipient.

In another aspect the present application provides process for preparation of crystalline Form A of Mavacamten, characterized by a PXRD pattern comprising peaks at about 11.5, 15.6, 17.2, 18.6, 19.9, 22.2, 23.3, 25.5, 29.0 and 31.5±0.2° 2θ, comprising:
a) providing a solution of Mavacamten,
b) adding an anti-solvent to the solution obtained in step (a),
c) optionally, heating the mixture of step (b), and
d) isolating crystalline Form A of Mavacamten.

The step (a) of the process involves preparation of a solution of Mavacamten in a suitable solvent such as dichloromethane, THF, methanol, ethanol, isopropanol or a mixture thereof. The mixture may be heated or sonicated to get clear solution.

The step (b) involves addition of an anti-solvent such as n-hexane, n-heptane, ethylacetate or diethyl ether. After adding the anti-solvent the resultant mixture may stirred for about 2 hours at 20° C. to about 50° C.

The step (c) involves isolation of crystalline Form A of Mavacamten. The crystalline Form A of Mavacamten is isolated from the suspension by filtration or by decantation or by any suitable method. The crystalline Form A of Mavacamten may be dried under vacuum.

In another aspect, the crystalline Form A of Mavacamten is further characterized by a PXRD pattern comprising the peaks at about 10.0, 11.5, 13.6, 14.5, 15.6, 16.1, 16.7, 17.2, 18.6, 19.9, 21.1, 22.2, 23.3, 23.7, 24.0, 24.4, 24.7, 25.5, 26.0, 27.2, 27.6, 29.0, 29.8, 31.5, 32.3, 32.6, 33.8, 34.6, 36.0, 36.4, 37.2, 37.8, 38.5 and 39.5±0.2° θ.

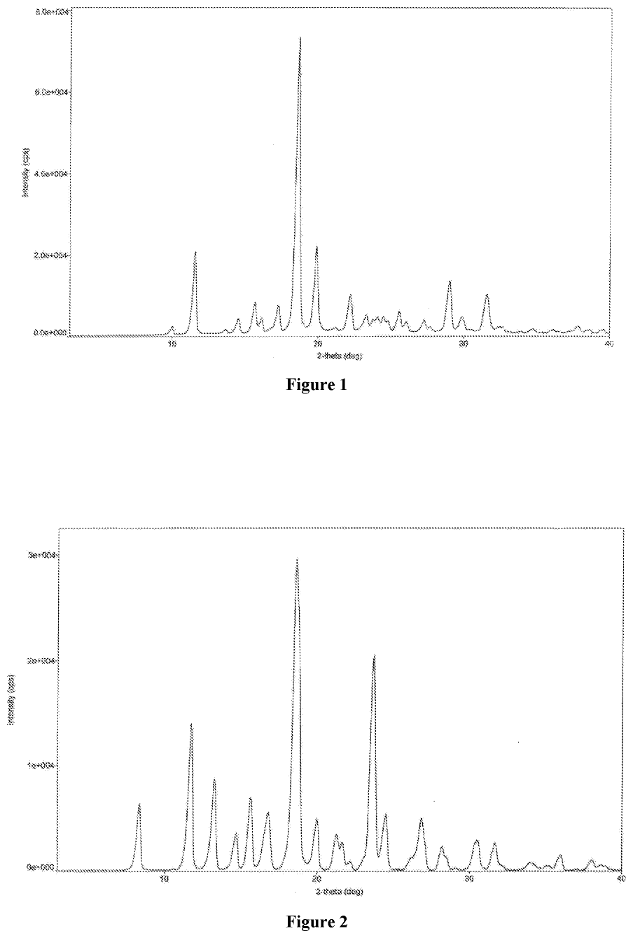

In another aspect, the crystalline Form A of Mavacamten is characterized by the PXRD pattern of FIG. 1.

In another aspect the present application provides a pharmaceutical composition comprising crystalline Form A of Mavacamten prepared by the process of the present application and a pharmaceutically acceptable excipient.

In another aspect the present application provides a process for preparation of crystalline Form B of Mavacamten characterized by a PXRD pattern comprising peaks at about 8.3, 11.7, 13.2, 15.6, 18.5, 18.7, 19.9, 22.1, 24.4 and 26.8±0.2° 2θ, comprising slurrying amorphous Mavacamten in water and isolating the crystalline Form B of Mavacamten.

In another aspect the present application provides a process for preparation of crystalline Form B of Mavacamten, comprising:
a) providing a solution of Mavacamten,
b) adding the solution obtained in step (a) into water at 0° C., and
c) isolating crystalline Form B of Mavacamten.

The step (a) of the process involves preparation of a solution of Mavacamten in a suitable solvent such as DMSO, DMF and THF. The mixture may be heated or sonicated to get clear solution.

The step (b) involves addition of an anti-solvent such as water. The solution may be cooled to 0° C. and water is added or water is first cooled to 0° C. and then the Mavacamten solution is added. After adding the anti-solvent the resultant mixture may stirred for about 2 hours at 0° C. to about 10° C.

The step (c) involves isolation of crystalline Form B of Mavacamten. The crystalline Form B of Mavacamten is isolated from the suspension by filtration or by decantation or by any suitable method. The wet solid may washed with a solvent such as n-hexane or ethylacetate. The crystalline Form B of Mavacamten may be dried under vacuum.

In another aspect, the crystalline Form B of Mavacamten is further characterized by a PXRD pattern comprising the peaks at about 8.3, 11.7, 13.2, 14.6, 15.6, 16.7, 18.5, 18.7, 19.9, 21.2, 21.6, 22.1, 23.6, 24.4, 26.2, 26.8, 28.1, 28.4, 29.0, 30.4, 31.6, 32.1, 34.0, 35.1, 35.9, 38.0 and 38.7±0.2° θ.

In another aspect, the crystalline Form B of Mavacamten is characterized by the PXRD pattern of FIG. 2.

In another aspect the present application provides a pharmaceutical composition comprising crystalline Form B of Mavacamten prepared by the processes of the present application and a pharmaceutically acceptable excipient.

In another aspect the present application provides a process for preparation of crystalline Form C of Mavacamten characterized by a PXRD pattern comprising peaks at about 7.8 and 18.1±0.2° 2θ, comprising:
a) providing a solution of Mavacamten in methanol,
b) optionally, heating the solution obtained in step (a) to 50° C., and
c) isolating crystalline Form C of Mavacamten.

The process involves preparation of a solution of Mavacamten in a methanol. The mixture may be heated or sonicated to get clear solution.

In one aspect the solution is heated to about 50° C. and stirred for about 10 minutes. The solution may be filtered to get rid of particles.

The clear solution is evaporated to get the crystalline Form C. The crystalline Form C of Mavacamten may be dried under vacuum.

In another aspect, the crystalline Form C of Mavacamten is further characterized by a PXRD pattern comprising the peaks at about 11.9 and 19.3° 2θ.

In another aspect, the crystalline Form C of Mavacamten is characterized by the PXRD pattern of FIG. 5.

In another aspect the present application provides a pharmaceutical composition comprising crystalline Form C of Mavacamten prepared by the processes of the present application and a pharmaceutically acceptable excipient.

In one embodiment, the present application provides process for preparation of crystalline Form D of Mavacamten characterized by a PXRD pattern comprising peaks at about 11.06, 14.4, 15.5, 16.9 and 19.1±0.2° 2θ, comprising:
a) heating Mavacamten to temperature up to 230° C.;
b) isolating crystalline Form D of Mavacamten.

In another embodiment, the present application provides a process for preparation of crystalline Form D of Mavacamten comprising heating Form A of Mavacamten to temperature from 180° C. to 230° C.

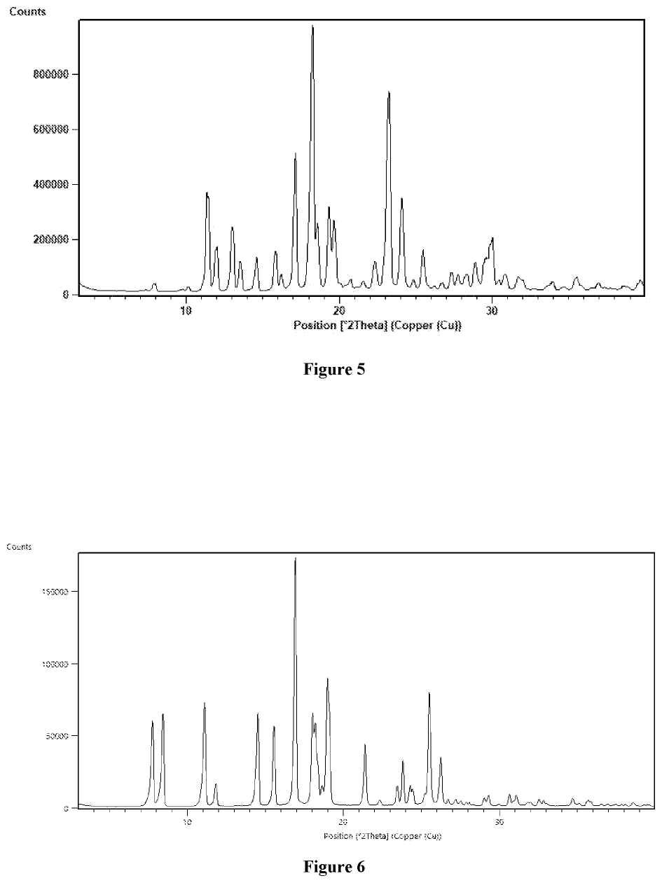

In another embodiment, the crystalline Form D of Mavacamten is characterized by the PXRD pattern of FIG. 6.

In another embodiment, the crystalline Form D of Mavacamten is characterized by the Differential scanning calorimetry (DSC) graph of FIG. 7.

In another embodiment, the present application provides pharmaceutical compositions comprising crystalline form D of Mavacamten prepared by the processes of the present application and one or more pharmaceutically acceptable excipient.

In one aspect the present application provides process for preparation of crystalline Form E of Mavacamten characterized by a PXRD pattern comprising peaks at about 6.39, 9.31, 13.87, 20.08 and 24.81±0.2° 2θ, comprising:
a) providing Mavacamten in one or more of suitable solvents;
b) isolating crystalline Form E of Mavacamten.

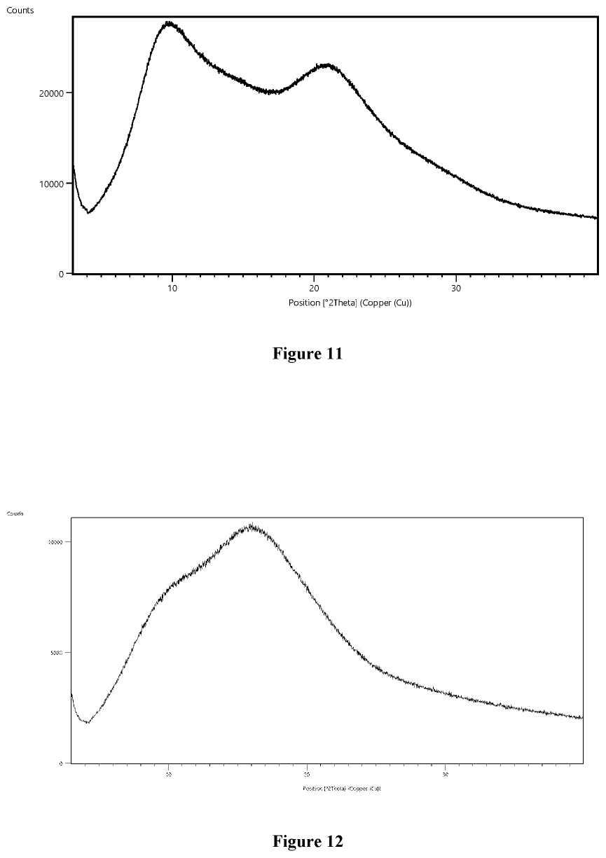

In another aspect the present application provides form E of Mavacamten, characterized by a PXRD pattern as represented by FIG. 11.

In another embodiment, the present application provides a process for preparation of crystalline form E of Mavacamten comprising providing amorphous form of Mavacamten in chloroform and isolating crystalline form E of Mavacamten.

In another embodiment, the present application provides pharmaceutical compositions comprising crystalline form E of Mavacamten prepared by the processes described in this application and one or more pharmaceutically acceptable excipient.

In another aspect the present application provides amorphous form of Mavacamten, characterized by a PXRD pattern as represented by FIG. 4.

In another aspect the present application provides pharmaceutical compositions comprising amorphous form of Mavacamten described in this application and one or more pharmaceutically acceptable excipient.

In another embodiment, crystalline form A, B, C, D and form E, and amorphous form of Mavacamten of the present invention or the pharmaceutical compositions thereof, comprises Mavacamten with a chemical purity of at least 99% by HPLC or at least 99.5% by HPLC or at least 99.9% by HPLC.

In another aspect the present application provides a process for preparation of Mavacamten having a purity greater than about 99.5% by HPLC, comprising:
a) reacting 6-chloro-3-isopropyl pyrimidine-2,4-dione of formula II or a salt thereof with at least 2.5 mole equivalents of (S)-(−)-α-methyl benzylamine of formula III in a suitable solvent to form crude Mavacamten,

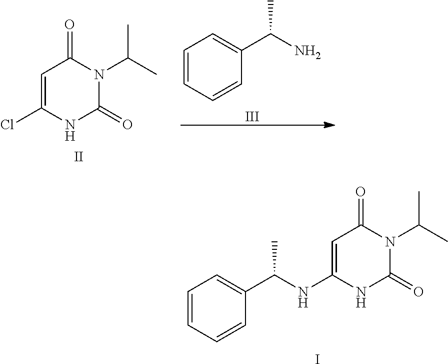

b) optionally, isolating the crude Mavacamten,
c) mixing the crude Mavacamten with water, and
d) isolating pure Mavacamten from the aqueous mixture.

The process involves reaction of compound of formula II with a compound of formula III in presence of a suitable solvent such as Dioxane, THF, Methyl-THF, ether and the like. The compound of formula II can be prepared by the processes described in the art. In one aspect, the present application provides that the compound of formula III is used at least 2.5 equivalents with respect to compound of formula II.

The compound of formula II, the solvent and the compound of formula III are mixed and the resulted mixture may be stirred for about 1 hour about 20 hours at a temperature of about 20° C. to about 100° C.

After completion of the reaction the reaction mixture is concentrated to yield crude Mavacamten. The crude Mavacamten is added to water, and the mixture may be extracted with a suitable solvent such as ethylacetate and the pure Mavacamten is isolated. Mavacamten prepared by this process has a purity of greater than 99.5% by HPLC, and contains less than about 0.1% of compound of formula II.

In another aspect, the present application provides a pharmaceutical composition comprising Mavacamten having a purity of greater than about 99.5% by HPLC and pharmaceutically acceptable excipient.

In another embodiment, the crystalline forms and solid dispersions of Mavacamten of the present invention are stable under thermal, humid and stress conditions.

In another embodiment, the crystalline forms and solid dispersions of Mavacamten of the present invention or the pharmaceutical compositions thereof, comprises Mavacamten with a chemical purity of at least 99% by HPLC or at least 99.5% by HPLC or at least 99.9% by HPLC.

In an embodiment, Mavacamten of present invention has average particle size of particles between 1 to 100 μm, less than 90 μm, less than 80 μm, less than 60 μm, less than 50 μm, less than 40 μm, less than 30 μm, less than 20 μm, less than 10 μm, less than 5 μm or any other suitable particle sizes. In another embodiment, Mavacamten of present invention may have particle size distribution: D10 of particles smaller than 20 μm, smaller than 15 μm, smaller than 10 μm, or smaller than 5 μm; D50 of particles smaller than 100 μm, smaller than 90 μm, smaller than 80 μm, smaller than 70 μm, smaller than 60 μm, smaller than 50 μm, smaller than 40 μm, smaller than 30 μm, smaller than 20 μm, smaller than 10 μm; D90 of particles smaller than 200 μm, smaller than 175 μm, smaller than 150 μm, smaller than 140 μm, smaller than 130 μm, smaller than 120 μm, smaller than 110 μm, smaller than 100 μm, smaller than 90 μm, smaller than 80 μm, smaller than 70 μm, smaller than 60 μm, smaller than 50 μm, smaller than 40 μm, smaller than 30 μm, smaller than 20 μm, smaller than 10 μm.

Particle size distributions of Mavacamten particles may be measured using any techniques known in the art. For example, particle size distributions of Mavacamten particles may be measured using microscopy or light scattering equipment, such as, for example, a Malvern Master Size 2000 from Malvern Instruments Limited, Malvern, Worcestershire, United Kingdom. As referred herein, the term “D10” in the context of the present invention is 10% of the particles by volume are smaller than the D10 value and 90% particles by volume are larger than the D10 value. “D50” in the context of the present invention is 50% of the particles by volume are smaller than the D50 value and 50% particles by volume are larger than the D50 value. “D90” in the context of the present invention is 90% of the particles by volume are smaller than the D90 value and 10% particles by volume are larger than the D90 value.

In an embodiment, Mavacamten of present invention can be micronized or milled using conventional techniques to get the desired particle size to achieve desired solubility profile to suit to pharmaceutical composition requirements. Techniques that may be used for particle size reduction include, but not limited to ball milling, roller milling and hammer milling. Milling or micronization may be performed before drying, or after the completion of drying of the product.

The compound of this application is best characterized by the X-ray powder diffraction pattern determined in accordance with procedures that are known in the art. X-ray diffraction was measured using PANalytical X-ray diffractometer, Model: Empyrean. Sytem description: CuK-Alpha 1 wavelength=1.54060, voltage 45 kV, current 40 mA, divergence slit=¼; Sample stage=Reflection-spinner. Revolution time [s]: 1.000; Scan type: Pre-set time; Detector—Pixcel; Measurement parameters: Start position [2Th.]: 3.0066; End Position [2Th.]: 39.9916; Step Size [2Th.]: 0.0130; Scan step time [s]: 1.000.

The chemical transformations described throughout the specification, may be carried out at ambient temperatures, but particular reactions may require the use of higher or lower temperatures, depending on reaction kinetics, yields, and the like. Furthermore, any of the chemical transformations may employ one or more compatible solvents, which may influence the reaction rates and yields. Depending on the nature of the reactants, the one or more solvents may be polar protic solvents, polar aprotic solvents, non-polar solvents, water or any of their combinations.

Suitable solvents to the reaction conditions include but are not limited to: alcohols, such as methanol, ethanol, 2-propanol, n-butanol, isoamyl alcohol and ethylene glycol; ethers, such as diisopropyl ether, dimethoxyethane, methyl tert-butyl ether, diethyl ether, 1,4-dioxane, tetrahydrofuran (THF), methyl THF, and diglyme; esters, such as ethyl acetate, isopropyl acetate, and t-butyl acetate and like; ketones, such as acetone and methyl isobutyl ketone and like; aliphatic hydrocarbons like n-hexane, cyclohexane, iso-octane and like; aromatic hydrocarbons like toluene, xylene and like; halogenated hydrocarbons, such as dichloromethane, dichloroethane, chloroform, and like; nitriles, such as acetonitrile; polar aprotic solvents, such as N,N-dimethylformamide, N,N-dimethylacetamide, N-methylpyrrolidone, dimethylsulfoxide, and the like; water; and any mixtures of two or more thereof.

The compounds obtained by the chemical transformations of the present application can be used for subsequent steps without further purification, or can be effectively separated and purified by employing a conventional method well known to those skilled in the art, such as recrystallization, column chromatography, by transforming them into a salt followed by optionally washing with an organic solvent or with an aqueous solution, and eventually adjusting pH. Compounds at various stages of the process may be purified by precipitation or slurrying in suitable solvents, or by commonly known recrystallization techniques. The suitable recrystallization techniques include, but are not limited to, steps of concentrating, cooling, stirring, or shaking a solution containing the compound, combination of a solution containing a compound with an anti-solvent, seeding, partial removal of the solvent, or combinations thereof, evaporation, flash evaporation, or the like. An anti-solvent as used herein refers to a liquid in which a compound is poorly soluble. Compounds can be subjected to any of the purification techniques more than one time, until the desired purity is attained.

Compounds may also be purified by slurrying in suitable solvents, for example, by providing a compound in a suitable solvent, if required heating the resulting mixture to higher temperatures, subsequent cooling, and recovery of a compound having a high purity. Optionally, precipitation or crystallization at any of the above steps can be initiated by seeding of the reaction mixture with a small quantity of the desired product. Suitable solvents that can be employed for recrystallization or slurrying include, but are not limited to: alcohols, such as, for example, methanol, ethanol, and 2-propanol; ethers, such as, for example, diisopropyl ether, methyl tert-butyl ether, diethyl ether, 1,4-dioxane, tetrahydrofuran (THF), and methyl THF; esters, such as, for example, ethyl acetate, isopropyl acetate, and t-butyl acetate; ketones, such as acetone and methyl isobutyl ketone; halogenated hydrocarbons, such as dichloromethane, dichloroethane, chloroform, and the like; hydrocarbons, such as toluene, xylene, and cyclohexane; nitriles, such as acetonitrile and the like; water; and any mixtures of two or more thereof.

The compounds at various stages of the process may be recovered using conventional techniques known in the art. For example, useful techniques include, but are not limited to, decantation, centrifugation, gravity filtration, suction filtration, evaporation, flash evaporation, simple evaporation, rotational drying, spray drying, thin-film drying, freeze-drying, and the like. The isolation may be optionally carried out at atmospheric pressure or under a reduced pressure. The solid that is obtained may carry a small proportion of occluded mother liquor containing a higher than desired percentage of impurities and, if desired, the solid may be washed with a solvent to wash out the mother liquor. Evaporation as used herein refers to distilling a solvent completely, or almost completely, at atmospheric pressure or under a reduced pressure. Flash evaporation as used herein refers to distilling of solvent using techniques including, but not limited to, tray drying, spray drying, fluidized bed drying, or thin-film drying, under atmospheric or a reduced pressure.

A recovered solid may optionally be dried. Drying may be suitably carried out using equipment such as a tray dryer, vacuum oven, air oven, fluidized bed dryer, spin flash dryer, flash dryer, and the like, at atmospheric pressure or under reduced pressure. Drying may be carried out at temperatures less than about 150° C., less than about 100° C., less than about 60° C., or any other suitable temperatures, in the presence or absence of an inert atmosphere such as nitrogen, argon, neon, or helium. The drying may be carried out for any desired time periods to achieve a desired purity of the product, such as, for example, from about 1 hour to about 15 hours, or longer.

### DEFINITIONS

The following definitions are used in connection with the present application unless the context indicates otherwise.

The term “about” when used in the present application preceding a number and referring to it, is meant to designate any value which lies within the range of ±10%, preferably within a range of ±5%, more preferably within a range of ±2%, still more preferably within a range of ±1% of its value. For example “about 10” should be construed as meaning within the range of 9 to 11, preferably within the range of 9.5 to 10.5, more preferably within the range of 9.8 to 10.2, and still more preferably within the range of 9.9 to 10.1.

All percentages and ratios used herein are by weight of the total composition and all measurements made are at about 25° C. and about atmospheric pressure, unless otherwise designated. All temperatures are in degrees Celsius unless specified otherwise. As used herein, “comprising” means the elements recited, or their equivalents in structure or function, plus any other element or elements which are not recited. The terms “having” and “including” are also to be construed as open ended. All ranges recited herein include the endpoints, including those that recite a range “between” two values. Whether so indicated or not, all values recited herein are approximate as defined by the circumstances, including the degree of expected experimental error, technique error, and instrument error for a given technique used to measure a value.

Certain specific aspects and embodiments of the present application will be explained in greater detail with reference to the following examples, which are provided only for purposes of illustration and should not be construed as limiting the scope of the application in any manner. Reasonable variations of the described procedures are intended to be within the scope of the present invention. While particular aspects of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

### EXAMPLES

### Example-1: Preparation of Mavacamten as Described in US '200

6-Chloro-3-isopropyl-pyrimidine-2,4-dione (2.0 g) and 1,4-dioxane (40 mL) were charged into a 100 mL round bottomed flask. (S)-(−)-α-methylbenzylamine (2.8 g) was added slowly. The mixture was heated to 80° C. and stirred for 24 hours. TLC showed that about 20% of starting material remain unreacted. The reaction mixture was concentrated under vacuum at 70° C. and then cooled to 25° C. Water (60 mL) was added, and the mixture obtained was stirred for 10 minutes, and then extracted with ethyl acetate (2×30 mL). The organic layer was separated and washed with 1N HCl (50 mL) and brine (50 mL) and concentrated under reduced pressure at 40° C.

30% ethyl acetate/hexane (40 mL) was added to the solid obtained and stirred for 15 minutes. The solid was filtered and washed with 30% ethyl acetate/hexane (20 mL), and dried first under suction, and then under vacuum for 2 hours at 25° C. Yield: 1.5 g (51.7%). Purity: 99.33% by HPLC. PXRD pattern is shown in FIG. 1.

### Example-2: Preparation of Pure Mavacamten

6-Chloro-3-isopropyl-pyrimidine-2,4-dione (80 g) and 1,4-dioxane (400 mL) were charged into a 2000 mL round bottomed flask. (S)-(−)-α-methylbenzylamine (154.2 g) was added slowly. The mixture was heated to 80° C. and stirred for 18 hours. TLC showed complete consumption of starting material. The reaction mixture was concentrated under vacuum at 80° C. and then cooled to 25° C. Water (1600 mL) was then added, the mixture obtained was then stirred for 15 minutes, and extracted with ethyl acetate (2×800 mL). The organic layer was separated and washed with 1N HCl (400 mL) and brine (800 mL).

The product which precipitated from the organic layer was filtered, washed with ethyl acetate (200 mL) and dried under vacuum at 40° C. Yield: 85 g (Crop 1), Yield: 73%. Purity: 99.84% by HPLC. PXRD pattern is shown in FIG. 1.

The filtrate was concentrated under vacuum at 40° C., 30% ethyl acetate/hexane (200 mL) was added to the solid and the mixture was stirred for 30 minutes. Filtered the solid and washed with 30% ethyl acetate/hexane (100 mL) and dried first under suction and the under vacuum for 2 hours at 25° C. Yield: 12 g (Crop 2).

### Example-3: Preparation of Mavacamten Form A

Mavacamten (400 mg), dichloromethane (8.5 mL) and methanol (1.5 mL) were charged into a 50 mL round bottomed flask. The mixture was sonicated till complete dissolution, and stirred for 30 minutes at 50° C. n-Hexane (50 mL) was added drop-wise over a period of 10 minutes. The resultant suspension was stirred for 2 hours at 50° C., then cooled to 28° C. and stirred for 30 minutes. The suspension was filtered and the solid obtained dried under vacuum to yield 360 mg of off-white solid. PXRD pattern is shown in FIG. 1.

### Example-4: Preparation of Mavacamten Form B

Mavacamten (350 mg) and DMSO (2 mL) were charged into a 50 mL round bottomed flask. The mixture was sonicated till complete dissolution. Water (50 mL) was charged into another 100 mL round bottomed flask and cooled 0° C. The Mavacamten DMSO solution was added drop-wise over a period of 10 minutes. The resultant suspension was stirred for 3 hours at 0° C. The suspension was filtered and the solid obtained was washed with n-hexane (50 mL). The solid was dried under vacuum for 1 hour to yield 290 mg of an off-white solid. PXRD pattern is shown in FIG. 2.

### Example-5: Preparation of Amorphous Solid Dispersion of Mavacamten and Polyvinylpyrrolidone K-30 (PVP K-30)

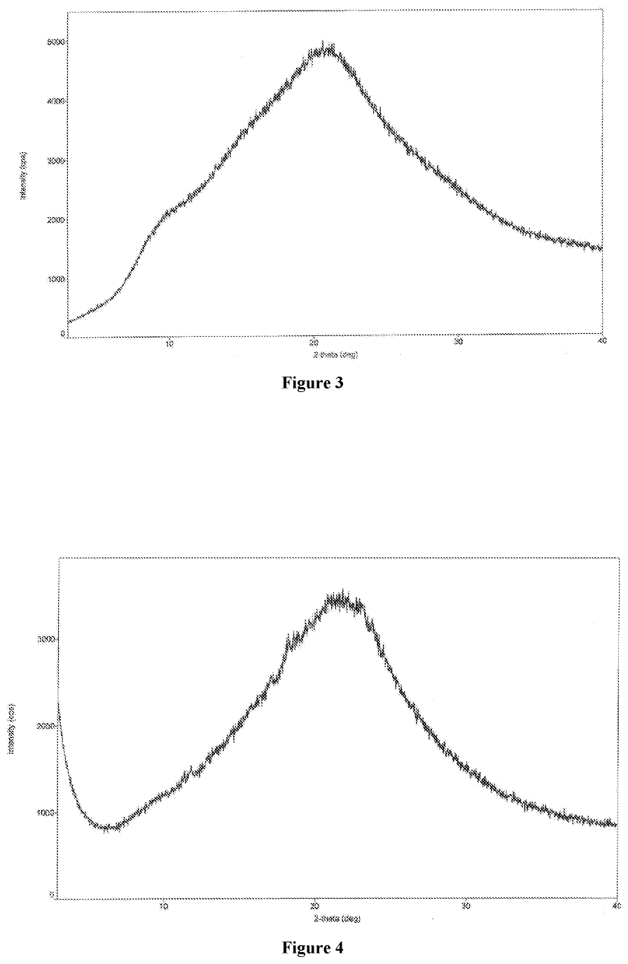

Mavacamten (250 mg), PINT K-30 (PVP K-30, 500 mg), methanol (2 mL) and dichloromethane (8 mL) were charged into a 50 mL conical flask at 27° C. The mixture was sonicated until complete dissolution. The solution obtained was filtered into a 50 mL round bottomed flask and concentrated under reduced pressure at 45° C. to afford a solid which was dried at 27° C. under reduced pressure to obtain 690 mg of amorphous solid dispersion. PXRD pattern is shown in FIG. 3.

### Example-6: Preparation of Amorphous Solid Dispersion of Mavacamten and Copovidone

Mavacamten (150 mg), Copovidone (300 mg), methanol (2 mL) and dichloromethane (18 mL) were charged into a 50 mL conical flask at 27° C. The mixture was sonicated until complete dissolution. The solution obtained was filtered into a 50 mL round bottomed flask and concentrated under reduced pressure at 45° C. to afford a solid which was dried at 27° C. under reduced pressure to obtain 420 mg of amorphous solid dispersion.

100 mg of above solid and Syloid 244 FP (50 mg) were charged into a mortar and pestle. The mixture was ground for 15 minutes to obtain free flowing solid. PXRD pattern is shown in FIG. 4.

### Example-7: Preparation of Crystalline Form C of Mavacamten

Mavacamten (200 mg) methanol (20 mL) were charged into a 50 mL round bottomed flask. The mixture was heated to 55° C. to get complete dissolution and the obtained solution was filtered into another 50 mL round bottom flask and the solvent was evaporated using rotavapor under vacuum at 55° C. The solid was dried at 27° C. under reduced pressure. PXRD pattern is shown in FIG. 5.

### Example-8: Preparation of Mavacamten Form D

Mavacamten Form A (50 mg) was heated up to 230° C. at a rate of 5° C./min and held at that temperature for about 10 min using TGA. Resulted material was checked for PXRD. PXRD pattern is shown in FIG. 1.

### Example-9: Preparation of Amorphous Form of Mavacamten

Mavacamten Form A (200 mg) was taken into a clean ball milling jar. The compound was ball milled for about 99 min. PXRD pattern is shown in FIG. 8.

### Example-10: Preparation of Amorphous Form of Mavacamten

Form A of Mavacamten (10 g) was dissolved in 500 mL of methanol and spray dried the solution at 60-70° C. of inlet temperature, flow rate of 8 g/min. Obtained spray dried material was checked for PXRD. The PXRD pattern is shown in FIG. 8.

### Example-11: Preparation of Mavacamten Form E

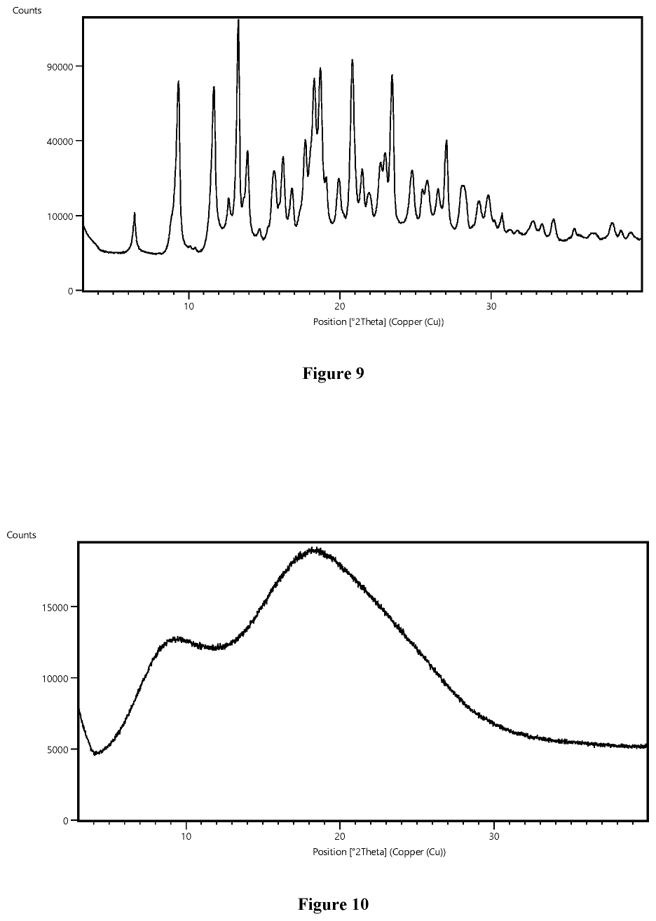

200 mg of amorphous form of Mavacamten was added to 3 mL of chloroform and the obtained slurry was sonicated for about 2 h. The slurry was filtered. Resulted material was checked for PXRD. PXRD pattern is shown in FIG. 9.

### Example-12: Preparation of Amorphous Solid Dispersion of Mavacamten with HPMC Phthalate

Mavacamten (200 mg) and of HPMC phthalate (800 mg) were dissolved in 50 mL of Methanol and 50 mL of acetone mixture, This solution was allowed to rapid solvent evaporation by rota vapor at 50-60° C. under vacuum, isolated material was collected and checked for PXRD. The PXRD pattern is shown in FIG. 10.

### Example-13: Preparation of Amorphous Solid Dispersion of Mavacamten with PVP K-90

Mavacamten (200 mg) and of PVP K-90 (800 mg) were dissolved in 35 mL of Methanol and 10 mL of acetone mixture. This solution was allowed to rapid solvent evaporation by rota vapor at 50-60° C. under vacuum, isolated material was collected and checked for PXRD. The PXRD pattern is shown in FIG. 11.

### Example-14: Preparation of Amorphous Solid Dispersion of Mavacamten with Eudragit L100-55

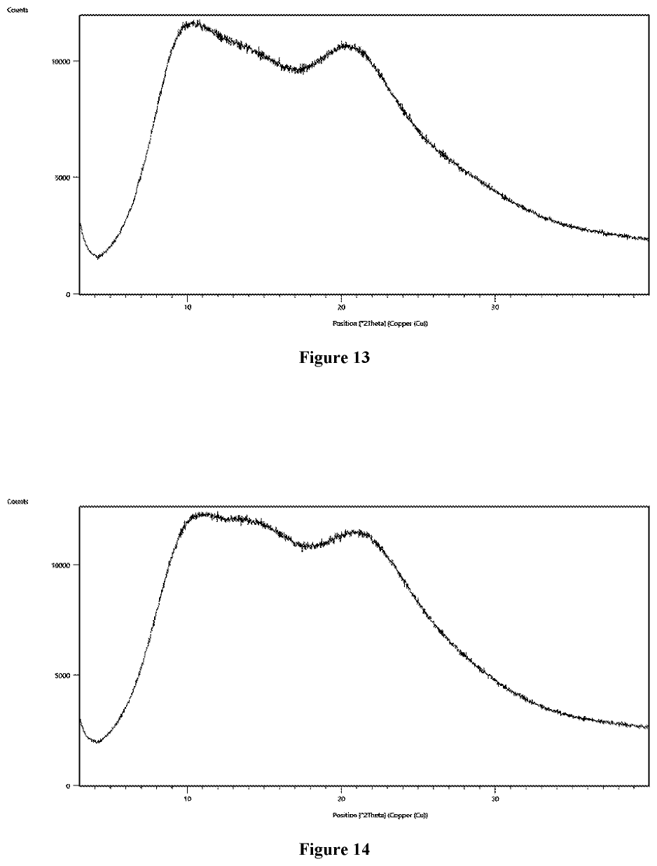

Mavacamten (200 mg) and Eudragit L100-55 (800 mg) were dissolved in 100 mL of Methanol and the solution was filtered under vacuum. The solution was allowed to rapid solvent evaporation by rotavapor at 60° C. under vacuum, isolated material was collected and checked for PXRD. The PXRD pattern is shown in FIG. 12.

### Example-15: Preparation of Amorphous Solid Dispersion of Mavacamten with PVP K-30

Mavacamten (200 mg), PVP K-30 (800 mg) and methanol (80 mL) were charged into a 250 mL Buchi flask at 27° C. The solution obtained was concentrated under reduced pressure at 55° C. to afford a solid which was dried at 25° C. under reduced pressure to obtain 600 mg of amorphous solid dispersion. PXRD pattern is shown in FIG. 13.

### Example-16: Preparation of Amorphous Solid Dispersion of Mavacamten and Copovidone

Mavacamten (200 mg), Copovidone VA64 (800 and methanol (80 mL) were charged into a 250 mL conical flask at 27° C. The mixture was stirred until complete dissolution. The solution obtained was filtered into a 250 mL Buchi flask and concentrated under reduced pressure at 55° C. to afford a solid which was dried at 25° C. under reduced pressure to obtain 700 mg of amorphous solid dispersion. PXRD pattern is shown in FIG. 14.

### Example-17: Preparation of Mavacamten Form B

Amorphous Mavacamten (1 g) and water (3 mL) were charged into a 50 mL round bottomed flask. The resultant suspension was stirred for 3 hours at 25° C. The suspension was filtered and dried under vacuum for 1 hour to yield 900 mg of an off-white solid. PXRD pattern is shown in FIG. 2.

1. Amorphous solid dispersion comprising Mavacamten and one or more pharmaceutically acceptable excipient.

2. The solid dispersion according to claim 1, wherein the pharmaceutically acceptable excipient is selected from the group comprising pregelatinized starch, lactose, powdered cellulose, microcrystalline cellulose, dicalcium phosphate, tricalcium phosphate, Polyethylene glycol, Copovidone, Soluplus, Silicified microcrystalline cellulose, mannitol, sorbitol, acacia, guar gum, tragacanth, gelatin, polyvinylpyrrolidone, hydroxymethyl celluloses, ethylcellulose, methylcellulose, hydroxypropyl cellulose, hydroxypropyl methylcellulose, HPMC-Phthalate, HPMC-AS, HPMC-15 CPS, sodium starch glycolate, crospovidone, croscarmellose sodium, colloidal silicon dioxide stearic acid, magnesium stearate, zinc stearate, colloidal silicon dioxide and mixtures thereof.

3. The solid dispersion according to claim 1, wherein the pharmaceutically acceptable excipient is selected from the group comprising Copovidone, PVP K-30, PVP K-90, HPMC Phthalate and Eudragit LI 00-55.

4. The solid dispersion according to claim 1, wherein the pharmaceutically acceptable excipient is Copovidone.

5. The solid dispersion according to claim 1, wherein the pharmaceutically acceptable excipient is Eudragit LI 00-55.

6. A process for preparing amorphous solid dispersion comprising Mavacamten and one or more pharmaceutically acceptable excipient, the process comprising;
(a) providing a solution comprising Mavacamten and one or more pharmaceutically acceptable excipients,
(b) removing solvent from the solution obtained in step (a), and
(c) recovering amorphous solid dispersion comprising Mavacamten and one or more pharmaceutically acceptable excipient.

7. The process according to claim 6, wherein the pharmaceutically acceptable excipient is selected from the group comprising Copovidone, PVP K-30, PVP K-90, HPMC Phthalate and Eudragit L100-55.

8. A pharmaceutical composition comprising the solid dispersion according to claim 1 and a pharmaceutically acceptable carrier.

9. A process for preparation of crystalline Form A of Mavacamten characterized by a PXRD pattern comprising peaks at about 11.5, 15.6, 17.2, 18.6, 19.9, 22.2, 23.3, 25.5, 29.0 and 31.5±0.2° 20, comprising:
a) providing a solution of Mavacamten in a suitable solvent,
b) adding an anti-solvent to the solution obtained in step (a),
c) optionally, heating the mixture of step (c), and
d) isolating crystalline Form A of Mavacamten.

10. The process of claim 9, wherein the suitable solvent is selected from the group comprising dichloromethane, methanol, ethanol, isopropanol and tetrahydrofuran or a mixture thereof.

11. The process of claim 9, wherein the anti-solvent is n-hexane, n-heptane, diethyl ether or ethylacetate.

12. A pharmaceutical composition comprising crystalline Form A of Mavacamten prepared by the process of claim 9 and a pharmaceutically acceptable carrier.

13. A process for preparation of crystalline Form B of Mavacamten characterized by a PXRD pattern comprising peaks at about 8.3, 11.7, 13.2, 15.6, 18.5, 18.7, 19.9, 22.1, 24.4 and 26.8±0.2° 20, comprising:
a) providing a solution of Mavacamten,
b) adding the solution obtained in step (a) into water at 0° C., and
c) isolating crystalline Form B of Mavacamten.

14. A process for preparation of crystalline Form B of Mavacamten characterized by a PXRD pattern comprising peaks at about 8.3, 11.7, 13.2, 15.6, 18.5, 18.7, 19.9, 22.1, 24.4 and 26.8±0.2° 20, comprising slurrying amorphous mavacamten in water and isolating crystalline Form B of Mavacamten.

15. A pharmaceutical composition comprising crystalline Form B of Mavacamten prepared by the process of claim 13 and a pharmaceutically acceptable carrier.

16. A process for preparation of crystalline Form C of Mavacamten characterized by a PXRD pattern comprising peaks at about 7.8 and 18.1±0.2° 20, comprising:
a) providing a solution of Mavacamten in methanol,
b) optionally, heating the solution obtained in step (a) to 50° C., and
c) isolating crystalline Form C of Mavacamten.

17. A process for preparation of amorphous form of Mavacamten, comprising:
a) providing Mavacamten in one or more of suitable solvents;
b) isolating amorphous form of Mavacamten.

18. A process for preparation of amorphous form of Mavacamten, comprising:
a) milling Mavacamten,
b) isolating amorphous form of Mavacamten.

19. The process according to claim 16, wherein the milling is ball milling.

20. A pharmaceutical composition comprising amorphous form of Mavacamten prepared by any of the process of claim 17 and a pharmaceutically acceptable carrier.

21. A pharmaceutical composition comprising crystalline Form B of Mavacamten prepared by the process of claim 14 and a pharmaceutically acceptable carrier.

22. A pharmaceutical composition comprising crystalline Form C of Mavacamten prepared by the process of claim 16 and a pharmaceutically acceptable carrier.

23. A pharmaceutical composition comprising amorphous form of Mavacamten prepared by any of the process of claim 19 and a pharmaceutically acceptable carrier.
